(19) United States
(12) Patent Application Publication
(10) Pub. No.: US 2010/0316588 A1
(43) Pub. Date: Dec. 16, 2010

(54) METHOD FOR COATING AN ELASTOMERIC MATERIAL WITH A LAYER OF ANTITOXIC MATERIAL

(76) Inventors: Pierre J. Messier, Quebec (CA); David Ohayon, Quebec (CA)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/765,755

(22) Filed: Apr. 22, 2010

Related U.S. Application Data
(60) Provisional application No. 61/214,312, filed on Apr. 22, 2009.

(51) Int. Cl. A01N 29/00; A61M 27/00; A41D 19/00; B32B 27/00; B32B 27/18; B32B 25/20; B32B 25/12; A61F 6/04; B05D 3/00; A01P 1/00

(52) U.S. Cl. 424/78.17; 604/544; 428/492; 428/521; 428/423.1; 428/493; 428/339; 428/334; 428/335; 428/340; 428/519; 2/167; 428/327; 428/411.1; 428/447; 428/422; 428/480; 428/475.5; 428/522; 428/220; 128/844; 427/299; 427/372.2; 427/2.3; 427/322

(57) ABSTRACT

The invention relates to elastomeric products that are coated with a thin layer of elastomeric polymeric coating containing an antitoxic agent, particularly a demand disinfectant iodinated resin. The antimicrobial coated catheters are prepared by adding the antitoxic agent to a solution of a liquid elastomeric polymer and then coating the surface of the elastomeric through a dipping or spraying procedure. The antimicrobial coatings can be applied to a variety of different elastomeric products including gloves and catheters and are capable of providing a high level of protection against microbes and other contaminants.

### CROSS-REFERENCE TO RELATED APPLICATION

This application claims priority to and the benefit of, and incorporates herein by reference in its entirety, U.S. Provisional Patent Application No. 61/214,312, which was filed on Apr. 22, 2009.

### BACKGROUND OF INVENTION

Elastomeric materials have proven to be very valuable in many healthcare and medicinal applications. Several types of elastomeric polymers have properties which are ideal for such applications. For instance, latex demonstrates a combination of softness, high tensile strength and excellent film-forming properties. Polyurethane, polyvinyl chloride (PVC), nitrile rubber, neoprene, and styrene-block copolymers also have beneficial properties. The choice of elastomer will be dependent on the desired application as well as other factors, including cost of manufacture.

Disposable elastomeric gloves are used in many healthcare related applications. These gloves are used to protect a wearer from contaminants including harmful microorganisms or contaminated biological fluids. The disposable gloves are usually generated from natural rubber latex, nitrile rubber, PVC or polyurethane. One significant problem with commercially available disposable gloves is that they often, during use, come in contact with exposed surfaces, potentially contaminating the surface. This is particularly an issue during surgeries, medical examinations and dental procedures where the gloves used by a doctor or dentist are exposed to dangerous microbes. Besides contaminating surfaces, there is the potential for cross-contamination of other patients and contamination of the doctor or dentist wearing the gloves.

When a glove is used in an environment such that it comes into contact with infectious pathogens or other dangerous contaminants, the addition of a coating containing an antimicrobial material reduces the risk of exposure to the infectious pathogens. However, developing such antimicrobial-coated gloves is challenging. Antimicrobial agents coated on elastomeric objects tend to rub off the surface of the glove, particularly when present in concentrations high enough to allow for efficient killing of microbes. Moreover, the presence of an antimicrobial agent may render the glove unusable. For example, the coating may compromise the durability or stretchability of the glove.

In addition to elastomeric gloves, other elastomeric materials benefit from antimicrobial coatings, including prophylactics (e.g. condoms) and catheters. The widespread use of respiratory catheters, venous and or arterial catheters and urological catheters has resulted in dangerous infections owing to the adherence and colonization of pathogens on the catheter surface. Moreover, colonized catheters may produce a reservoir of resistant microorganisms. Catheter-associated urinary tract infections are now the most common type of hospital acquired infection. Catheter-related bloodstream and respiratory infections are also very common and often result in morbidity. Antimicrobial catheters currently on the market have been shown to offer some degree of protection against dangerous microbes. These catheters use various active agents such as ionic silver, chlorhexidine and antibiotics. However, commercially available antimicrobial catheters have considerable drawbacks including a narrow range of activity and the potential to cause undesirable side effects. Furthermore, development of bacterial resistance against these active agents is quite common, rendering them ineffective.

Hence, there is a need to develop new antimicrobial products, such as gloves and catheters, that are effective against a large array of microorganisms, are nontoxic and are inexpensive to manufacture.

### SUMMARY OF INVENTION

A new method of manufacturing gloves and catheters coated with antimicrobial agents is described herein. The methodology involves coating an elastomeric glove or catheter with a thin layer comprising an antimicrobial agent stably dispersed within an elastomeric matrix. In preferred embodiments, the antimicrobial agent is a demand disinfectant iodinated resin.

The coating process may be performed without (or with minimal) application of heat, thereby avoiding deactivation of the antimicrobial agent, yet still achieving stable adherence of the coating to the glove or catheter. Further, it is found that a very thin coating containing an iodinated resin as antibacterial agent is sufficient to achieve excellent antimicrobial properties without adversely impacting the performance properties of the product (e.g., flexibility and strength). The elastomeric glove or catheter may be made from the same or a different elastomer than the elastomeric coating (e.g., the product and/or the coating may each or separately contain latex, nitrile rubber, polyurethane, polyvinyl chloride (PVC), neoprene, styrene, silicone, styrene block copolymer, polytetrafluoroethylene (Teflon®), nylon, etc.). In certain embodiments, the product foundation and coating are advantageously composed of the same elastomer. The iodinated resin serves as an antimicrobial agent which prevents or greatly inhibits hazardous microbes that the gloves or catheters contact from spreading to any surfaces or liquids that are touched.

The invention relates to elastomeric products that are coated with a thin layer of elastomeric polymeric coating containing an antitoxic agent, particularly a demand disinfectant iodinated resin. The antimicrobial-coated catheters are prepared by adding the antitoxic agent to a solution of a liquid elastomeric polymer and then coating the surface of the elastomeric product through a dipping or spraying procedure. The antimicrobial coatings can be applied to a variety of different elastomeric products, including gloves catheters, prophylactics and elastomeric films, and are capable of providing a high level of protection against microbes and other contaminants.

In one aspect, the invention is directed to an elastomeric product with enhanced antimicrobial properties, the product comprising: a foundation comprising an elastomeric material; and a coating applied over the foundation, the coating comprising iodinated resin particles stably dispersed within an elastomeric matrix. In certain embodiments, the elastomeric matrix of the coating comprises natural latex, synthetic latex, nitrile rubber (nitrile butadiene rubber, NBR), and/or polyurethane. In certain embodiments, the product is a glove, a catheter, or a prophylactic (e.g., condom).

In certain embodiments, the coating and/or the foundation comprises latex. The coating may advantageously have a thickness in the range from 5 μm to 250 μm, or from 20 μm to 100 μm, or from 50 μm to 80 μm, or from 65 μm to 75 μm, for example—this may be particularly advantageous where the coating comprises latex. The product may advantageously have a surface iodinated resin concentration in the range from 1 g/m², to 50 g/m² from 2 g/m² to 20 g/m², from 3 g/m² to 10 g/m², or from 5 g/m² to 7 g/m², for example—this may be particularly advantageous where the coating comprises latex.

In certain embodiments, the coating and/or the foundation comprises nitrile rubber. The coating may advantageously have a thickness in the range from 5 μm to 80 μm, or from 10 μm to 80 μm, or from 15 μm to 50 μm, or from 20 μm to 30 μm, for example—this may be particularly advantageous where the coating comprises nitrile rubber. The product may advantageously have a surface iodinated resin concentration in the range from 1 g/m² to 50 g/m², from 2 g/m² to 10 g/m², from 2 g/m² to 6 g/m², or from 3 g/m² to 4 g/m², for example—this may be particularly advantageous where the coating comprises nitrile rubber.

In certain embodiments, the iodinated resin particles advantageously have an average size within the range from 1 μm to 20 μm or within the range from 4 μm to 10 μm.

In certain embodiments, the coating comprises silicone, polyvinyl chloride, neoprene, styrene, styrene block copolymer, polyethylene, polytetrafluoroethylene (Teflon®), and/or nylon.

In another aspect, the invention is directed to a method for preparing a coated product with enhanced antimicrobial properties, the method comprising the steps of: (a) providing a foundation on a form of the product, the foundation comprising an elastomeric material; (b) optionally, applying a solvent to the foundation which would remove an existing coating of the foundation and/or prepare the surface for secondary treatment; (c) preparing a coating mixture comprising iodinated resin particles stably dispersed within a liquid elastomeric matrix; and (d) applying the coating mixture to the foundation and allowing the coating mixture to dry, all without heating the coating mixture, or with heating the coating at a temperature below about 160° C. for no more than about 20 minutes. In certain embodiments the coating is not heated above 150° C., 130° C., 100° C., or 90° C. In certain embodiments, the coating is not heated for longer than 15 minutes, 10 minutes, or 5 minutes. In certain embodiments, the coated product is a glove, a catheter, or a prophylactic (e.g., a condom).

In certain embodiments, step (d) comprises spraying the coating mixture onto the foundation. In certain embodiments, step (d) comprises dipping the foundation into the coating mixture.

In certain embodiments, where the foundation comprises nitrile rubber, the coating mixture comprises nitrile rubber, the coating has thickness in the range from 10 μm to 80 μm, the iodinated resin particles have an average size within the range from 4 μm to 20 μm, and the coating has an iodinated resin concentration in the range from 2 wt. % to 25 wt. %. In certain embodiments, where the foundation comprises latex, the coating mixture comprises latex, the coating has thickness in the range from 20 μm to 100 μm, the iodinated resin particles have an average size within the range from 4 μm to 20 μm, and the coating has an iodinated resin concentration in the range from 2 wt. % to 25 wt. %.

In certain embodiments, the concentration of iodinated resin particles in the coating mixture is in the range from 2 wt. % to 25 wt. %; in the range from 5 wt. % to 15 wt. %, or in the range from 7 wt. % to 13 wt. %.

In another aspect, the invention is directed to an elastomeric film with enhanced antimicrobial properties, the film comprising iodinated resin particles stably dispersed within an elastomeric matrix. The elastomeric matrix may comprise natural latex, synthetic latex, nitrile rubber, polyurethane, silicone, polyvinyl chloride, neoprene, styrene, styrene block copolymer, polyethylene, polytetrafluoroethylene, and/or nylon. The film may advantageously have thickness in the range from 5 μm to 250 μm, from 20 μm to 100 μm, or from 50 μm to 80 μm. The iodinated resin particles may have an average size within the range from 1 μm to 20 μm, or from 4 μm to 10 μm. The concentration of iodinated resin particles in the film may be in the range from 2 wt. % to 25 wt. %, or from 5 wt. % to 15 wt. %.

In yet another aspect, the invention is directed to a medical glove or catheter made from an elastomeric polymer which is coated with a thin layer of an elastomeric polymer containing iodinated resin particulates. The coating provides a significant amount of protection against a broad array of biocidal agents and other contaminants.

Another aspect of the present invention is directed to antimicrobial coatings for elastomeric products comprising an elastomeric polymer selected from the group consisting of latex, nitrile rubber, or polyurethane and a plurality of iodinated resin particles incorporated in the elastomeric polymer, wherein the thickness of the coating is in the range from about 20 μm to about 100 μm.

In yet another aspect, the present invention provides a new method of manufacturing gloves and/or catheters coated with a thin layer of an elastomeric polymer containing an antitoxic agent. The methodology involves coating the glove or catheter, formed of an elastomeric polymer (e.g. latex or nitrile rubber), with a coating solution comprising a demand disinfectant iodinated resin stably dispersed within a liquid solution of the same type or a different type of elastomeric polymer as the glove or catheter.

Elements of embodiments described with respect to a given aspect of the invention may be used in various embodiments of another aspect of the invention (e.g., subject matter of dependent claims may apply to more than one independent claim).

### BRIEF DESCRIPTION OF FIGURES

FIG. 1 is a graph showing biological performance of liquid latex/iodinated resin coated latex elastomers of the present invention against the challenge microorganism Pseudomona aeruginosa.

FIG. 2 is a graph showing biological performance of liquid latex/iodinated resin coated latex elastomers of the present invention against the challenge microorganism S. aureus MRSA.

FIG. 3 is a graph showing biological performance of the liquid latex/iodinated resin coated latex elastomers against various challenge microorganisms including Pseudomona. aeruginosa, S. aureus MRSA, and Influenza A (H1N1).

FIG. 4 is a graph showing biological performance of the liquid latex/iodinated resin coated latex elastomers of the present invention against the challenge microorganism Pseudomona. aeruginosa.

FIG. 5 is a graph showing biological performance of antimicrobial coated catheters of the present invention compared to prior art antimicrobial catheters.

### DETAILED DESCRIPTION OF THE INVENTION

The following sections describe exemplary embodiments of the present invention. It should be apparent to those skilled in the art that the described embodiments of the present invention provided herein are illustrative only and not limiting, having been presented by way of example only.

Throughout the description, where items are described as having, including, or comprising one or more specific components, or where processes and methods are described as having, including, or comprising one or more specific steps, it is contemplated that, additionally, there are items of the present invention that consist essentially of, or consist of, the one or more recited components, and that there are processes and methods according to the present invention that consist essentially of, or consist of, the one or more recited processing steps.

It should be understood that the order of steps or order for performing certain actions is immaterial, as long as the invention remains operable. Moreover, two or more steps or actions may be conducted simultaneously. Scale-up and/or scale-down of systems, processes, units, and/or methods disclosed herein may be performed by those of skill in the relevant art. Processes described herein are configured for batch operation, continuous operation, or semi-continuous operation.

The present invention relates generally to elastomeric products, such as medical gloves, catheters, prophylactics and elastomeric films that are coated with a layer of elastomeric material incorporated with an antitoxic material, and methods of making the same. The antitoxic agent is preferably an antimicrobial agent, an antiviral agent, a bio-chemical agent or a reducing agent. The active agent preferably exerts a toxic effect on a diverse array of microorganisms and other pathogens and environmental toxins while not being toxic to the user. Preferably, the antitoxic agent comprises iodinated resin particles. Other active agents that may be used in addition to—or, in alternative embodiments, instead of—the iodinated resin include, but are not limited to, triclosan, diatomic halogens, silver, copper, zeolyte with an antimicrobial attached thereto, halogenated resins, and agents capable of devitalizing/deactivating microorganisms/toxins that are known in the art, including for example activated carbon, other metals and other chemical compounds. The purpose of the antitoxic agent is to provide an enhanced barrier of protection to the elastomeric while reducing the risk of exposure to infectious pathogens in both healthcare and non-healthcare settings.

Iodine/resin demand disinfectants are known in the art. For example, U.S. Pat. No. 5,639,452 (“the '452 patent”), to Messier, the entire contents which are hereby incorporated by reference, describes a process for preparing an iodine demand disinfectant resin from an anion exchange resin. The demand disinfectant iodinated resins described in the '452 patent may be ground into a powder. One preferred demand disinfectant iodinated resin is Triosyn® brand iodinated resin powders made by Triosyn Research Inc., a division of Triosyn Corporation of Vermont, USA. The particle sizes of the powders range from about 1 micron to about 150 microns. Preferably, the particle sizes should be in the range from about 4 microns to about 10 microns.

Triosyn® iodinated resin powders used in accordance with the present invention are referred to as Triosyn® T-50 iodinated resin powder, Triosyn® T-45 iodinated resin powder, Triosyn® T-40 iodinated resin powder or Triosyn® T-35 iodinated resin powder. The base polymer used to manufacture such iodinated resins is Amberlite® 4020H (Rohm & Haas). These resins contain quaternary ammonium exchange groups with are bonded to styrenedivinyl benzene polymer chains. Other base polymers could be used. The numbers refer to the approximate weight percentage of iodine relative to the resin. Powders with other weight percentages of iodine may also be used in accordance with the present invention. Different percentages of iodine in the iodinated resin powders will confer different properties to the powder, in particular, different levels of biocidal activity. The particular resin used is based on the desired application. It is important to note that iodinated resin from other sources can also be used.

In a preferred embodiment of the present invention, a Triosyn® iodinated resin powder is mixed with a liquid elastomeric polymer such as liquid latex, liquid nitrile rubber, or liquid polyurethane, for a period of time sufficient to incorporate the powder into the liquid polymer. The concentration of Triosyn® iodinated resin powder in the liquid elastomeric polymer may vary from about 2% to about 25% by weight, and is preferably in the range from about 10% to about 15% by weight. When fully incorporated, the resultant solution can be sprayed onto the surface of an elastomeric material. Alternatively, the elastomeric coating may be applied by dipping the elastomeric material in the liquid polymer solution. After drying, the elastomeric material will contain a uniform coating of elastomeric polymer with the Triosyn® iodinated resin powder incorporated therein.

In one embodiment of the present invention, the methodology described in the preceding paragraph is applied to the coating of an elastomeric glove. The underlying glove to be coated may be made from any suitable elastomeric material. Preferably, the glove is made from synthetic or natural latex. The glove may also be made from other elastomeric polymers including but not limited to nitrile rubber, neoprene, polyurethane, polyvinyl chloride, or a styrene-block copolymer. The underlying glove may be made from traditional methods well-known in the art. For example, the underlying glove may be formed by dipping a hand-shaped form coated with coagulant into a solution of liquid latex. The resultant latex glove is removed from the solution, dried and subsequently vulcanized. It is important to note that this process can be adapted to obtain varying thickness. Alternatively, the underlying glove to be coated may be any commercially available elastomeric glove. In this case, it is generally preferable to remove any preexisting coating on the glove because such a coating may decrease the adherence of the antimicrobial coating to the underlying elastomeric surface.

The antimicrobial coating made in accordance with the present invention can be applied to the glove through a spraying or dipping procedure, resulting in adherence of the antimicrobial coating to the surface of the underlying elastomeric glove. The underlying product foundation may comprise the same elastomeric material as the coating. Alternatively, the product foundation may be made of a different elastomeric material than the coating.

In a preferred embodiment of the present invention, the antimicrobial coating comprises a Triosyn® iodinated resin powder incorporated in liquid latex. However, other liquid elastomeric materials may be used in place of liquid latex, such as liquid nitrile rubber or liquid polyurethane. As discussed in the examples below, the Triosyn® iodinated resin powder is incorporated into the liquid elastomeric polymer by stirring until fully dispersed within the elastomeric matrix. The Triosyn® iodinated resin powder may have an average particle size in the range from 1 to 20 μm, and preferably in the range from 4 to 10 μm. The antimicrobial solution may then be sprayed onto the underlying elastomer and dried. Alternatively, the underlying elastomeric material may be dipped into the antimicrobial solution and then dried. Both techniques generate a product with a thin elastomeric coating (e.g., latex coating) in which the Triosyn® iodinated resin powder is embedded within the elastomeric matrix. The iodinated resin may be incorporated in the interstitial pores of the elastomeric coating and/or chemically bonded thereto.

The antimicrobial iodinated resin-containing liquid latex coatings preferably have a thickness in the range of 5 μm to 250 μm, preferably in the range of 20 μm to 100 μm, more preferably in the range of 50 μm to 80 μm and most preferably in the range of 65 μm to 75 μm. The percent weight increase of the glove upon application of the latex coating ranges from about 10% to about 70%. In preferred embodiments, the iodinated resin concentration of the coating is chosen within a range from about 1 g/m² to about 50 g/m², preferably from about 3 g/m² to about 10 g/m² and most preferably from about 5 g/m² to about 7 g/m². The antimicrobial iodinated resin containing liquid nitrile rubber coatings preferably have a thickness in the range of 10 μm to 150 μm, more preferably in the range of 15 μm to 50 μm and most preferably in the range of 20 μm to 30 μm. The percent weight increase of the glove upon application of coating ranges from about 10% to about 70%. The iodinated resin concentration of the nitrile coating ranges from about 2 g/m² to about 6 g/m², and preferably from about 3 g/m² to about 4 g/m².

Generally, in order to ensure strong adhesion of a coating to the underlying elastomeric material, the coated material is heated following the spraying or dipping procedure. However, in the presence of an antimicrobial agent, such heating may result in leeching of the antimicrobial agent and/or degradation of the antimicrobial agent. We have found that when the antimicrobial/liquid latex solution is sprayed onto an underlying latex glove, the resultant antimicrobial-coated gloves can be dried at room temperature and still adhere very strongly to the underlying latex surface. The strong adhesion between the two latex layers is likely the result of strong intermolecular interactions between the layers. As a result of the process, the Triosyn® iodinated resin powder has long-term stability, does not appreciably leech, and is not chemically degraded.

In another embodiment of the present invention, a small amount of heating may be applied to ensure adhesion between the underlying elastomeric surface and the elastomeric coating. For example, if the elastomeric coating and the underlying elastomeric material are made of different materials, heating may be required to ensure strong binding between the layers.

The methodology described in the preceding paragraphs allows for very strong adherence of the coating to the underlying latex material. Hence, the glove may have the appearance of being comprised of a single continuous layer. Because the antimicrobial coated layer is relatively thin, the coating does not compromise the stretchability or durability of the glove. Moreover, the resultant antimicrobial gloves retain their tactile feel and have excellent gripping properties.

In another embodiment of the present invention, the antimicrobial solutions containing iodinated resin powder can be applied to the surface of a catheter. The underlying catheter surface to be coated is preferably comprised of latex, silicone, polyvinyl chloride, polyurethane, polyethylene, Teflon®, nylon, or a mixture thereof. Similar to embodiments with the gloves, a solution of an iodinated resin in liquid polymer is sprayed onto the underlying catheter surface. Alternatively, the catheter can be dipped into the antimicrobial solution containing iodinated resin in the liquid polymer. Preferred coatings include latex and nitrile rubber. The properties of the coating, including thickness and concentration of iodinated resin, are similar to those described above for elastomeric gloves. As with the coated gloves described above, the underlying catheter may be comprised of the same or different material as the polymeric material used in the coating. The antimicrobial catheters of the present invention prevent adherence and colonization of pathogens on the catheter surface due to the added antimicrobial properties of the iodinated resin. Hence, the catheters of the present invention significantly reduce the development of catheter-associated urinary tract, respiratory and bloodstream infections, without compromising the performance of the catheter for its intended use.

As discussed in the Background section, a particular problem often faced with antimicrobial coated elastomeric gloves and catheters is that the biocidal material may leech from the surface of the elastomeric product. Hence, the antimicrobial efficacy is significantly reduced over time. Moreover, such leeching may create significant problems, particularly if the elastomeric products are used in medical or dental applications. A significant advantage of the present invention is that the iodinated resin powders incorporated in the coating do not have a tendency to rub off of the surface of the glove. For example, no Triosyn® iodinated resin powder was observed to leech following exposure to water, 70% alcohol gel, or white cellulose paper.

Another significant advantage of the present invention is that a relatively small amount of the antimicrobial agent need be applied in order to exert a significant toxic effect on a broad spectrum of pathogens. Unlike methods in the prior art, in which the antimicrobial agent is directly incorporated into the underlying elastomeric material, the present invention involves incorporating the antimicrobial agent only into the relatively thin outer coating layer. As such, the amount of antimicrobial agent needed to exert a toxic effect is significantly lessened. Clearly, this methodology also is advantageous from both a cost and manufacturing perspective.

With regards to efficacy, the elastomeric materials of the present invention have been tested on several challenge organisms and show remarkable activity (see Results section, below). For example, the antimicrobial-coated elastomeric materials of the present invention show greater than a 99.9999% reduction against gram-positive and gram-negative (P. aeruginosa) at contact exposure times as short as two minutes. Results obtained with Triosyn® iodinated resin powder suggest a consistent dose-dependent antimicrobial effect.

The methodology described above for producing antimicrobial-coated gloves and catheters may also be used to coat a host of other articles such as prophylactics, stents, and tubing.

The following examples illustrate various aspects and embodiments of the present invention. They are not to be construed to limit the claims in any manner whatsoever.

### Methods of Coating Gloves

### Preparing Glove to be Coated

1) Take a ceramic form and wrap the bottom of the form with paper towel (or other material) to prevent latex solution from being sprayed directly onto it.
2) Place a commercially available latex glove, which is powder-free and chlorinated, onto a ceramic form.
3) Spray toluene or Methyl Ethyl Ketone (MEK) or another type of organic solvent onto a paper towel (or other material) and carefully wipe the glove, especially in between the fingers, to remove any existing coating from the glove. This will increase the adherence of the new latex coating onto the glove foundation.
4) Let the toluene on the gloves evaporate at room temperature in the fume hood.

### Preparing the Coating Formulation

1) In a plastic weigh boat, carefully weigh the appropriate amount of 3 μm Triosyn® T50 powder needed for the desired concentration and for a particular total solution size. A Triosyn® particle of 10 μm could also be used, for example.
   i. For example: a 75 g latex solution containing 15% w/w of Triosyn® T50 in purple latex, one would have to weigh 11.25 g of powder.
2) In a stainless steel container, add a stir bar and carefully weigh the appropriate amount of liquid latex of any color.
   i. For example: for a 75 g total solution size containing 15% w/w of Triosyn® T-50 powder, one would have to weigh 63.75 g of latex.
3) Place the stainless steel container with the liquid latex on a stir plate and start stirring the latex until a good vortex can be seen in the middle (600 rpm—medium).
4) Start to slowly incorporate the Triosyn® iodinated resin powder into the liquid latex, making sure the solution always has a good vortex in the middle. The rpm of the stirring should be gradually increased until it reaches approximately 1000 to 1100 rpm.
5) When the whole amount of Triosyn® iodinated resin powder has been added, let the solution stir for 10 minutes at 1000-1100 rpm.

### Spraying the Coating On the Glove

1) Having already cleaned and prepared the nozzle of the spray gun, set the air pressure to about 75 psi to ensure a uniform coating.
2) To ensure the good working status of the spray gun, dip the feed tube in a beaker filled with water and spray some water to make sure nothing is clogging the system.
3) Adjust the setting at the front left side of the nozzle to dispense the widest possible spray.
4) Remove the spray gun from the water beaker and spray the remainder of the water present in the system.
5) Attach the stainless steel container to the nozzle of the spray gun, making sure both parts are carefully attached to each other.
6) Spray a small quantity of the latex solution to ensure once more that the system is free of particles.
7) Take the form with a clean glove on and start to gently spray the fingers from all angles to ensure a uniform coating.
8) With all or most of the fingers coated, start to coat the palm, the back of the hand, as well as the cuff.
9) Spray over the various regions to give a thick enough and uniform coating.
10) Let the coating dry at room temperature. Drying can be expedited by using a fan.
11) When dried, wash the exterior and interior of the glove in warm water for about 2 minutes and then allow the excess water to flow off and allow to dry the glove to dry at room temperature.

### Methods of Coating Catheters

### Preparing Catheter to be Coated

1) Take a commercially available catheter and soak it in SU100 Silicone Remover for about 5 hours to ensure the complete removal of added coating on the base polymeric material.
2) Rinse the catheter under water to remove all of the SU100 solution and allow it to completely dry at room temperature.
3) When dried, remove all additional coatings to reach the base polymeric material and ensure that the surface of the catheter is free of particles.
4) Place a rod (metal or plastic) in the middle of the catheter to allow for more rigidity during the spray coating.

Following preparation of the catheter to be coated, the coating solution is prepared and applied to the catheter surface in identical fashion as described above with respect to gloves.

### EXPERIMENTAL RESULTS

The following results show the microbiological data obtained using coated antimicrobial gloves manufactured using the process described above.

### A. Biological Testing Against Different Challenge Organisms

The following method was used to test the antimicrobial efficacy of the antimicrobial gloves of the present invention against different challenge microorganisms. Tests were performed using the liquid inoculum AATCC 100 Test Method (Assessment of Antibacterial Finishes on Textile Materials). In the test, Triosyn® iodinated resin coated gloves or catheters (i.e., Triosynated samples) of size swatches of 1″×1″ produced in accordance with the present invention were exposed to a sample of a liquid microbial suspension for contact times of 1, 2 or 5 minutes. The sample was then placed in a neutralizing fluid to recover viable microorganisms and the viable microorganisms were counted. Examples 1-5 show the results of various biological tests.

### Example 1

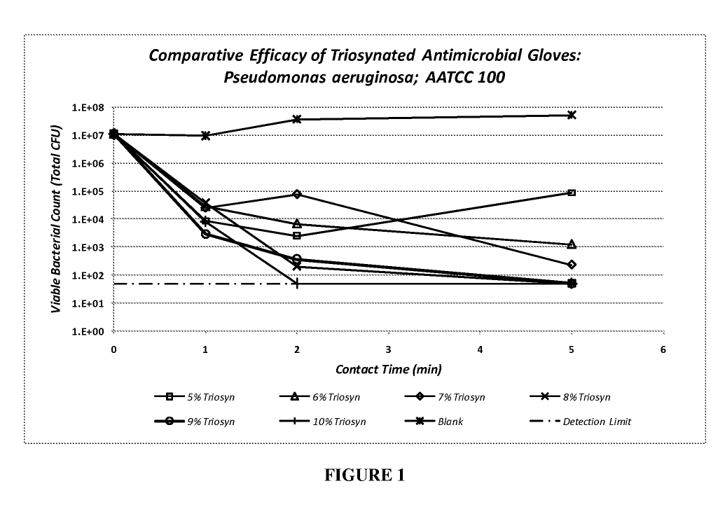

Latex gloves (Kimberley Clark Latex glove (Product code: SP 2330)) coated with a solution of iodinated resin powder (Triosyn® T50 powder) (4 micron) in liquid latex were prepared using methods described above. The concentrations of Triosyn® T-50 iodinated resin powder in the liquid latex were varied between 5 and 10% by weight. The challenge organism was P. aeruginosa. Results at time periods from 0 minutes to 5 minutes are displayed in Table 1 and graphically depicted in FIG. 1. The antimicrobial-coated materials show a greater than 99.9999% reduction of P. aeruginosa at contact exposure times as short as two minutes for certain concentrations of iodinated resin.

TABLE 1
Antimicrobial Performance against Pseudomonas aeruginosa

| Contact | Blank (n = 3) | Glove + 5% Triosyn (n = 3) | | Glove + 6% Triosyn (n = 3) | | Glove + 7% Triosyn (n = 3) | |
|---|---|---|---|---|---|---|---|
| Time | (CFU Total) | (CFU Total) | % Reduction | (CFU Total) | % Reduction | (CFU Total) | % Reduction |
| 0 min | 1.10E+07 | N/A | N/A | N/A | N/A | N/A | N/A |
| 1 min | 9.40E+06 | 8.70E+03 | 99.91% | 2.80E+04 | 99.75% | 2.51E+04 | 99.73% |
| 2 min | 3.67E+07 | 2.50E+03 | 99.99% | 6.73E+03 | 99.98% | 7.67E+04 | 99.79% |
| 5 min | 5.17E+07 | 8.65E+04 | 99.83% | 1.23E+03 | 99.998% | 2.33E+02 | 99.9995% |
| Contact | Blank (n = 3) | Glove + 8% Triosyn (n = 3) | | Glove + 9% Triosyn (n = 3) | | Glove + 10% Triosyn (n = 3) | |
| Time | (CFU Total) | (CFU Total) | % Reduction | (CFU Total) | % Reduction | (CFU Total) | % Reduction |
| 0 min | 1.10E+07 | N/A | N/A | N/A | N/A | N/A | N/A |
| 1 min | 9.40E+06 | 3.88E+04 | 99.38% | 2.98E+03 | 99.97% | 8.17E+03 | 99.91% |
| 2 min | 3.67E+07 | 2.00E+02 | 99.9995% | 3.67E+02 | 99.999% | <5.00E+01 | >99.999907% |
| 5 min | 5.17+E+07 | <5.00E+01 | >99.999933% | <5.00E+01 | >99.999933% | <5.00E+01 | >99.999933% |

Detection level = 50 CFU

### Example 2

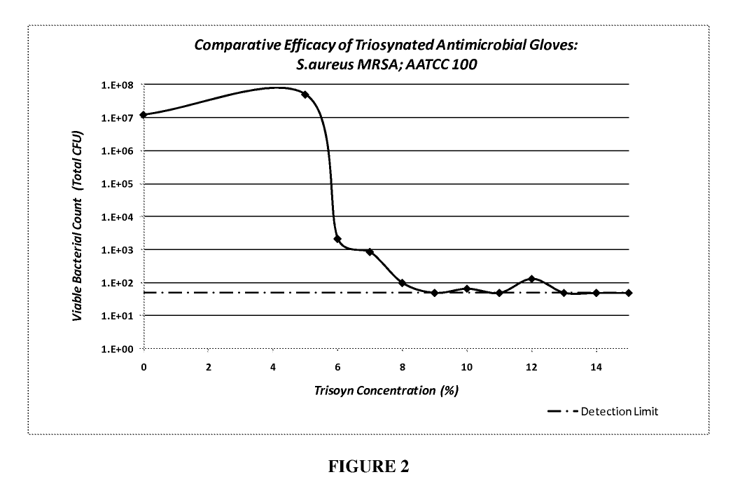

Experiments as described in Example 1 were repeated with the challenge organism being S. aureus MRSA. Triosyn® T-50 iodinated rein powder concentrations in liquid latex were varied between 5 and 15% by weight. The samples were tested after a time period of 2 minutes. Results are displayed in Table 2 and are graphically depicted in FIG. 2. The antimicrobial-coated elastomeric materials of the present invention shows a greater than 99.99995% reduction of S. aureus MRSA at contact a exposure time as short as two minutes.

TABLE 2
Antimicrobial Performance against S. aureus MRSA at
A Contact Time of 2 Minutes

| | |
|---|---|
| | S. aureus MRSA Counts |
| Triosyn | (n = 3) |

| Concentration (%) | CFU Total | % Reduction |
|---|---|---|
| 0 | 1.22E+07 | N/A |
| 5 | 5.03E+07 | 99.5874% |
| 6 | 2.17E+03 | 99.9822% |
| 7 | 8.67E+02 | 99.9929% |
| 8 | 1.00E+02 | 99.9992% |
| 9 | 5.00E+01 | 99.9996% |
| 10 | 6.67E+01 | 99.9995% |
| 11 | 5.00E+01 | 99.9996% |
| 12 | 1.33E+02 | 99.9989% |
| 13 | <5.00E+01 | >99.999590% |
| 14 | <5.00E+01 | >99.999590% |
| 15 | <5.00E+01 | >99.999590% |

Detection level = 50 CFU

### Example 3

Experiments described in Examples 1 and 2 were repeated but with different color coating additives. Table 3 shows the effect of different color coating additives on biological performance with the challenge organism being P. aeruginosa. The concentration of iodinated resin in these tests was 15% by weight in liquid latex and contact time was 2 minutes. As can be seen from Table 3, the presence of coating additives did not appreciably affect biological performance.

TABLE 3
Effect of different color coatings additives;
Antimicrobial Performance against Pseudomonas aeruginosa

| | Contact Time | | |
|---|---|---|---|
| | 0 min | | |
| Glove | (n = 3) | 2 min (n = 3) | |
| Treatment | (CFU Total) | (CFU Total) | % Reduction |
| Clear Coating Blank | 9.00E+06 | 7.92E+06 | 12.04% |
| Clear Coating + Triosyn | N/A | <1.67E+01 | >99.999789% |
| Black Coating Blank | 9.32E+06 | 1.45E+07 | 0.00% |
| Black Coating + Triosyn | N/A | 1.67E+01 | 99.9999% |
| Green Coating Blank | 1.05E+07 | 1.33E+07 | 0.00% |
| Green Coating + Triosyn | N/A | 1.67E+01 | 99.9999% |
| Purple Coating Blank | 1.29E+07 | 1.23E+07 | 5.04% |
| Purple Coating + Triosyn | N/A | 1.67E+01 | 99.9999% |
| Orange Coating Blank | 1.19E+07 | 2.29E+07 | 0.00% |
| Orange Coating + | N/A | 1.67E+01 | 99.9999% |
| Triosyn | | | |
| Red Coating Blank | 1.64E+07 | 2.05E+07 | 0.00% |
| Red Coating + Triosyn | N/A | 3.33E+01 | 99.9998% |

### Example 4

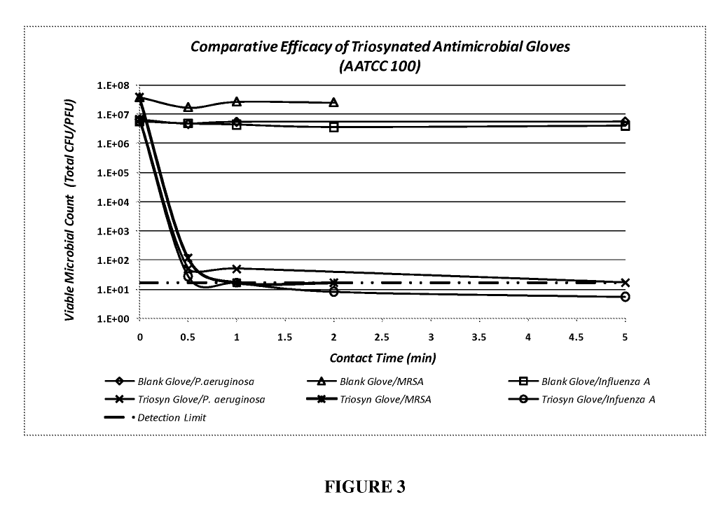

Following the excellent results obtained in experiments described above, the antimicrobial gloves of the present invention were tested on several challenge organisms. Accordingly, the AATCC test method was used to demonstrate the efficacy of the gloves against the challenge organisms. In these experiments, the latex gloves were coated with a 15% solution of Triosyn® T-50 powder (4 micron) in liquid latex. As shown in Tables 4-6, a greater than 99.999% reduction was demonstrated against Gram-positive (S. aureus MRSA) (Table 5) and Gram-negative bacteria (P. aeruginosa) (Table 4), and influenza virus (Table 6) exposed to contact times as short as thirty seconds for Triosyn-treated latex gloves. The results from Tables 4-6 are graphically depicted in FIG. 3.

TABLE 4
Antimicrobial Performance against Pseudomonas aeruginosa

| Contact | Blank (n = 6) | Latex Glove + 15% Triosyn (n = 6) | | |
|---|---|---|---|---|
| Time | (CFU Total) | (CFU Total) | Log Reduction | % Reduction |
| 0 | 6.51E+06 | N/A | N/A | N/A |
| 30 sec | 4.62E+06 | <5.00E+01 | >4.97 | >99.998917% |
| 1 min | 5.43E+06 | <5.00E+01 | >5.04 | >99.999080% |
| 5 min | 5.55E+06 | <1.67E+01 | >5.52 | >99.999700% |

Detection level = 16.7 CFU

TABLE 5
Antimicrobial Performance against Staphylococcus aureus MRSA

| Contact | Blank (n = 6) | Latex Glove + 15% Triosyn (n = 6) | | |
|---|---|---|---|---|
| Time | (CFU Total) | (CFU Total) | Log Reduction | % Reduction |
| 0 | 3.73E+07 | N/A | N/A | N/A |
| 30 sec | 1.70E+07 | 1.17E+02 | 5.30 | 100.00% |
| 1 min | 2.65E+07 | <1.67E+01 | >6.20 | >99.999937% |
| 2 min | 2.48E+07 | <1.67E+01 | >6.17 | >99.999933% |

Detection level = 16.7 CFU

TABLE 6
Antimicrobial Performance against Influenza A (H1N1)

| | | |
|---|---|---|
| | | Latex Glove + 15% |
| Contact | Blank (n = 3) | Triosyn (n = 3) |

| Time | (PFU Total) | (PFU Total) | % Reduction |
|---|---|---|---|
| 0 | 5.72E+06 | N/A | N/A |
| 30 sec | 4.78E+06 | 2.78E+01 | 99.99940% |
| 1 min | 4.39E+06 | <1.67E+01 | >99.999620% |
| 2 min | 3.56E+06 | <8.33E+00 | >99.999766% |
| 5 min | 4.00E+06 | <5.56E+00 | >99.999861% |

Detection level = 16.7 PFU

### Example 5

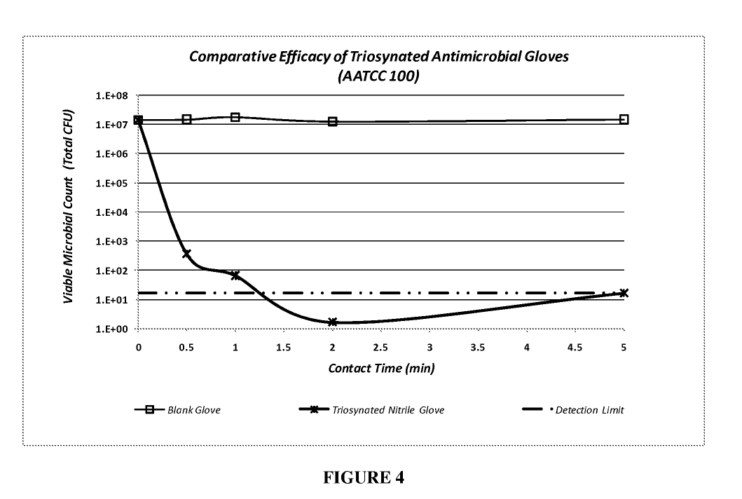

The tests described above were repeated on the challenge organism P. aeruginosa but with nitrile rubber gloves (Cardinal Health Nitrile powder free exam gloves (Product code: 8812N medium)) coated with a 15% solution of Triosyn® T-50 powder (4 micron) in liquid nitrile rubber. Results are shown in Table 7 below. As shown in Table 7, a 99.999% reduction was demonstrated against Gram-negative bacteria (P. aeruginosa) exposed to contact times as short as thirty seconds for iodinated resin treated gloves. These results are graphically depicted in FIG. 4.

TABLE 7
Antimicrobial Performance AgainstPseudomonas
aeruginosa for liquid nitrile rubber/iodinated resin coated elastomer

| Contact | Blank (n = 6) | Nitrile Glove + 15% Triosyn (n = 3) | | |
|---|---|---|---|---|
| Time | (CFU Total) | (CFU Total) | Log Reduction | % Reduction |
| 0 | 1.42E+07 | N/A | N/A | N/A |
| 30 sec | 1.46E+07 | 3.67E+02 | 5.03 | 99.9990% |
| 1 min | 1.76E+07 | 6.67E+01 | 5.45 | 99.9996% |
| 2 min | 1.26E+07 | <1.67E+00 | >5.88 | >99.999868% |
| 5 min | 1.47E+07 | <1.67E+01 | >5.94 | >99.999886% |

Detection level = 16.7 CFU

### B. Biological Testing of Antimicrobial Coated Elastomers Formed by Different Methods

Antimicrobial performance was evaluated with two different manufacturing processes of the current invention, dipping and spraying. The challenge microorganism employed in these studies was P. auruginosa. A latex coating containing iodinated resin was employed in the two studies. Hence, the methods involved either spraying the iodinated resin/liquid latex solution or dipping the latex gloves in the iodinated resin/liquid latex solution. Biological performance of the sprayed and dipped samples are shown in Tables 8 and 9, respectively. Consistent antimicrobial performance was demonstrated with the two manufacturing processes (spraying vs. dipping).

TABLE 8
Latex Gloves Sprayed With Triosyn Solution

| Contact | Blank (n = 6) | Latex Glove + 15% Triosyn (n = 6) | | |
|---|---|---|---|---|
| Time | (CFU Total) | (CFU Total) | Log Reduction | % Reduction |
| 0 | 6.51E+06 | N/A | N/A | N/A |
| 30 sec | 4.62E+06 | <5.00E+01 | >4.97 | >99.998917% |
| 1 min | 5.43E+06 | <5.00E+01 | >5.04 | >99.999080% |
| 5 min | 5.55E+06 | <1.67E+01 | >5.52 | >99.999700% |

Detection level = 16.7 CFU

TABLE 9
Gloves Dipped in Solution Containing Triosyn

| Contact | Blank (n = 6) | Latex Glove + 15% Triosyn (n = 6) | | |
|---|---|---|---|---|
| Time | (CFU Total) | (CFU Total) | Log Reduction | % Reduction |
| 0 | 5.37E+00 | N/A | N/A | N/A |
| 30 sec | 2.53E+06 | 8.33E+01 | 4.58 | 99.9967% |
| 1 min | 5.92E+06 | 1.83E+01 | 4.69 | 99.9969% |
| 5 min | 5.10E+06 | <1.67E+01 | >5.49 | >99.999673% |

Detection level = 16.7 CFU

### C. Zone of Inhibition Studies—Iodinated Resin Coated Catheters

The antimicrobial efficacy of the iodinated resin coated catheters (latex) of the present invention were determined using the bacterial challenge, Staphylococcus aureus ATCC 6538. Small segments of the iodinated resin coated catheter or a control catheter (no iodinated resin) were place on 1 cm² swatches of duct tape in an agar plate containing the challenge organism. After the required incubation time, the inhibition zone represented by a clear zone in the bacterial lawn surrounding the antimicrobial-containing article was readily obtained. A zone of inhibition is a region of the agar plate where the bacteria stop growing. The more sensitive the microbes are to the test article, the larger the zone of inhibition. In the two studies, the control catheter did not show a zone of inhibition whereas the iodinated resin coated catheter showed a zone of inhibition of 3 mm.

### D. Antimicrobial Properties of Iodinated Resin Coated Catheters

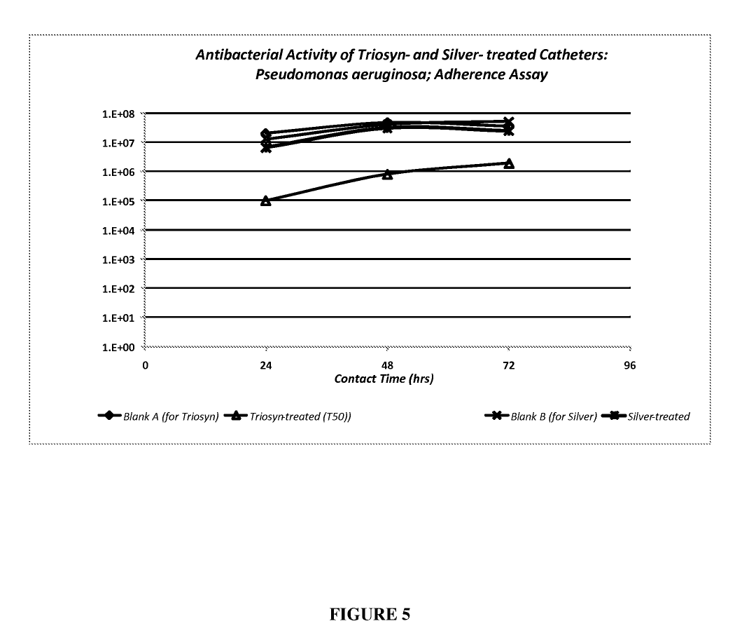

The antimicrobial efficacy of the antimicrobial catheters of the present invention was determined using a bacterial adherence assay (Jansen B. et al. “In-vitro efficacy of a central venous catheter complexed with iodine to prevent bacterial colonization” Journal of Antimicrobial Chemotherapy, 30:135-139, 1992). Accordingly, iodinated resin coated catheter (latex)-pieces were incubated in bacterial suspensions of P. aeruginosa for contact times of 24, 48, 72 or 96 hours followed by enumeration of adherent bacteria on the catheters using the colony count method. All iodinated resin coated catheters were coated with a 15% Triosyn solution of Triosyn® T-50 powder (4 micron) in liquid latex. Control experiments were run either with untreated (blank) catheters or commercially available silver-treated latex catheters (Bardex I. C. with Bard hydrogel and Bacti-Guard silver alloy coating). Results of these experiments are shown in Tables 10 and 11 and depicted graphically in FIG. 5.

The results of the study indicate that the iodinated resin-coated catheters (with Triosyn® T50) inhibited the adherence of bacteria for the duration of the test. On the other hand, silver-treated catheters showed little inhibitory effect on bacterial adherence.

TABLE 10
Antibacterial Activity of Iodinated Resin Coated
Catheters Over a 72 Hour Period against P. aeruginosa

| | |
|---|---|
| | Catheter + Triosyn |
| Blank (n = 3) | T50 (n = 3) |

| Contact | Viable Count | Viable Count | |
|---|---|---|---|
| Time | (CFU Total) | (CFU Total) | % Reduction |
| 24 hrs | 1.97E+07 | 9.90E+04 | 99.498% |
| 48 hrs | 4.75E+07 | 7.92E+05 | 98.333% |
| 72 hrs | 3.47E+07 | 1.88E+06 | 94.577% |

Detection level = 50 CFU

TABLE 11
Antibacterial Activity of Silver Treated Catheters
Over a 72 Hour Period against P. aeruginosa

| | |
|---|---|
| Blank (n = 3) | Catheter + Silver* (n = 3) |

| Contact | Viable Count | Viable Count | |
|---|---|---|---|
| Time | (CFU Total) | (CFU Total) | % Reduction |
| 24 hrs | 1.28E+07 | 6.43E+06 | 49.870% |
| 48 hrs | 3.95E+07 | 2.99E+07 | 24.219% |
| 72 hrs | 5.02E+07 | 2.34E+07 | 53.355% |

Detection level = 50 CFU
*Bardex I.C. with Bard Hydrogel and Bacti-Guard Silver Alloy Coating

### EQUIVALENTS

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. An elastomeric product with enhanced antimicrobial properties, the product comprising:
a foundation comprising an elastomeric material; and
a coating applied over said foundation, said coating comprising iodinated resin particles stably dispersed within an elastomeric matrix.

2. The product according to claim 1, wherein the elastomeric matrix of the coating comprises a member selected from the group consisting of natural latex, synthetic latex, nitrile rubber (nitrile butadiene rubber, NBR), and polyurethane.

3. The product according to claim 2, wherein the coating comprises latex.

4. The product according to claim 3, wherein the foundation comprises latex.

5. The product according to claim 3, wherein the coating has thickness in the range from 5 μm to 250 μm.

6. The product according to claim 5, wherein the coating has thickness in the range from 50 μm to 80 μm.

7. The product according to claim 6, wherein the coating has thickness in the range from 65 μm to 75 μm.

8. The product according to claim 3, wherein the product has a surface iodinated resin concentration in the range from 1 g/m² to 50 g/m².

9. The product according to claim 8, wherein the product has a surface iodinated resin concentration in the range from 5 g/m² to 7 g/m².

10. The product according to claim 2, wherein the coating comprises nitrile rubber.

11. The product according to claim 10, wherein the foundation comprises nitrile rubber.

12. The product according to claim 10, wherein the coating has thickness in the range from 5 μm to 80 μm.

13. The product according to claim 12, wherein the coating has thickness in the range from 15 μm to 50 μm.

14. The product according to claim 13, wherein the coating has thickness in the range from 20 μm to 30 μm.

15. The product according to claim 10, wherein the product has a surface iodinated resin concentration in the range from 1 g/m² to 50 g/m².

16. The product according to claim 15, wherein the product has a surface iodinated resin concentration in the range from 3 g/m² to 4 g/m².

17. The product according to claim 1, wherein the product is a glove.

18. The product according to claim 1, wherein the product is a catheter.

19. The product according to claim 1, wherein the iodinated resin particles have an average size within the range from 1 μm to 20 μm.

20. The product according to claim 1, wherein the iodinated resin particles have an average size within the range from 4 μm to 10 μm.

21. The product according to claim 1, wherein the coating comprises a member selected from the group consisting of silicone, polyvinyl chloride, neoprene, styrene, styrene block copolymer, polyethylene, polytetrafluoroethylene (Teflon®), and nylon.

22. A method for preparing a coated product with enhanced antimicrobial properties, the method comprising the steps of:
(a) providing a foundation on a form of the product, the foundation comprising an elastomeric material;
(b) optionally, applying a solvent to the foundation which would remove an existing coating of the foundation and/or prepare the surface for secondary treament
(c) preparing a coating mixture comprising iodinated resin particles stably dispersed within a liquid elastomeric matrix; and
(d) applying the coating mixture to the foundation and allowing the coating mixture to dry, all without heating the coating mixture, or with heating the coating at a temperature below about 160° C. for no more than about 20 minutes.

23. The method of claim 22, wherein step (d) comprises spraying the coating mixture onto the foundation.

24. The method of claim 22, wherein step (d) comprises dipping the foundation into the coating mixture.

25. The method of claim 22, wherein the coated product is a glove.

26. The method of claim 22, wherein the coated product is a catheter.

27. The method of claim 22, wherein the foundation comprises nitrile rubber, the coating mixture comprises nitrile rubber, the coating has thickness in the range from 10 μm to 80 μm, the iodinated resin particles have an average size within the range from 4 μm to 20 μm, and the coating has an iodinated resin concentration in the range from 2 wt. % to 25 wt. %.

28. The method of claim 22, wherein the foundation comprises latex, the coating mixture comprises latex, the coating has thickness in the range from 20 μm to 100 μm, the iodinated resin particles have an average size within the range from 4 μm to 20 μm, and the coating has an iodinated resin concentration in the range from 2 wt. % to 25 wt. %.

29. The method of claim 22, wherein the concentration of iodinated resin particles in the coating mixture is in the range from 2 wt. % to 25 wt. %.

30. The method of claim 22, wherein the concentration of iodinated resin particles in the coating mixture is in the range from 5 wt. % to 15 wt. %.

31. The product according to claim 1, wherein the product is a prophylactic.

32. An elastomeric film with enhanced antimicrobial properties, the film comprising iodinated resin particles stably dispersed within an elastomeric matrix.

33. The film of claim 32, wherein the elastomeric matrix comprises a member selected from the group consisting of natural latex, synthetic latex, nitrile rubber, polyurethane, silicone, polyvinyl chloride, neoprene, styrene, styrene block copolymer, polyethylene, polytetrafluoroethylene, and nylon.

34. The film of claim 32, wherein the film has thickness in the range from 5 μm to 250 μm.

35. The film of claim 34, wherein the film has thickness in the range from 20 μm to 100 μm.

36. The film of claim 32, wherein the iodinated resin particles have an average size within the range from 1 μm to 20 μm.

37. The film of claim 36, wherein the iodinated resin particles have an average size within the range from 4 μm to 10 μm.

38. The film of claim 32, wherein the concentration of iodinated resin particles in the film is in the range from 2 wt. % to 25 wt. %.

39. The film of claim 38, wherein the concentration of iodinated resin particles in the film is in the range from 5 wt. % to 15 wt. %.
